(19) United States
(12) Patent Application Publication
(10) Pub. No.: US 2008/0167500 A1
(43) Pub. Date: Jul. 10, 2008

(54) Process for the Preparation of a Diastereomerically Enriched Compound

(75) Inventors: Quirinus Bernardus Broxterman, Munstergeleen (NL); Ben De Lange, Munstergeleen (NL); Henrius Leonardus Marie Elsenberg, Tegelen (NL); Matthias Weber, Regensburg (DE)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 11/632,699

(22) PCT Filed: Jul. 20, 2005
(86) PCT No.: PCT/EP05/07988

§ 371 Date: Mar. 6, 2008

(30) Foreign Application Priority Data
Jul. 22, 2004 (EP) 04077123.0

(51) Int. Cl. C07C 211/01

(52) U.S. Cl. 564/453

(57) ABSTRACT

The present invention relates to a process for the preparation of a diastereomerically enriched compound, wherein a first compound according to formula (I), is contacted with a second compound according to formula (II), to form a third compound according to formula (III), whereby the compound according to formula (III) is subsequently reduced and thereby converted into a compound according to formula (IV), in which formulas: R₁=a cycloalkyl group whereby R₁ # R₂, R₂=a substituted or unsubstituted: (cyclo)alkyl group, (cyclo)alkenyl group, aryl group, cyclic or acyclic heteroalkyl group or heteroaryl group, R₃=an alkyl group, R₄=a substituted or unsubstituted: phenyl- or naphthyl-group, *=a chiral center. The invention furthermore relates to a diastereomerically enriched compound according to formula (IV) and its use in the preparation of pharmaceutical and agrochemically active compounds. The invention further relates to a process for the preparation of enantiomerically enriched compounds of formula (V), through hydrogenolysis of diastereomerically enriched compounds of formula (IV), wherein R₁ and R₂ have the meanings given above.

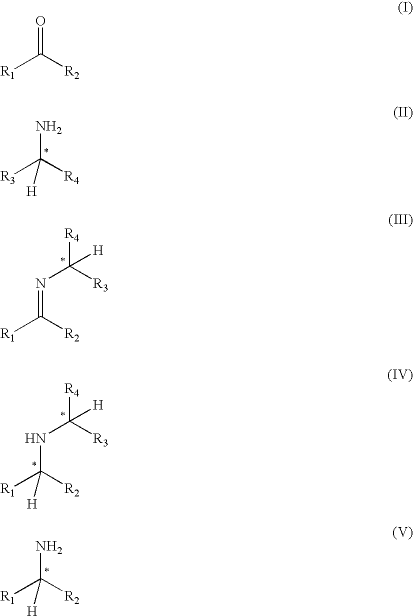

The present invention relates to a process for the preparation of a diastereomerically enriched compound and said diastereomerically enriched compound.

A process for the preparation of a diastereomerically enriched compound, containing substituted or non-substituted cycloalkyl groups, is disclosed by Pedrosa et al, J. Org. Chem, 1996, 61, pages 4130-4135. In this disclosure a stereoselective ring opening of chiral 1,3-oxazolidines by Grignard or organoaluminum reagents is described as key step in the synthesis of enantiopure cycloalkylamines. Drawback is the use of an expensive chiral auxiliary, (−)-8-benzylamino menthol, which needs to be prepared in two steps from (+)-pulegone. Furthermore, due to its high cost the chiral auxiliary must be recycled. Moreover, handling of Grignard and/or organoaluminum reagents that are air and moisture sensitive makes this process less suitable for industrial production.

Disadvantage of the process as described by Pedrosa is that it hardly is suitable for industrial production.

Object of the present invention is to provide a process for the preparation of a diastereomerically enriched compound containing substituted or non-substituted cycloalkyl groups that is suitable for industrial production.

This object is achieved with a process wherein a first compound according to formula I

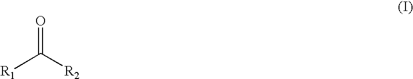

in which formula
R₁=a cycloalkyl group whereby R₁≠R₂
R₂=a substituted or unsubstituted: (cyclo)alkyl group, (cyclo)alkenyl group, aryl group, cyclic or acyclic heteroalkyl group or heteroaryl group,
   is contacted with an enantiomerically enriched compound according to formula II

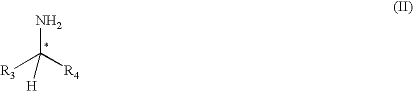

in which formula
R₃=an alkyl group
R₄=a substituted or unsubstituted: phenyl- or naphthyl-group
*=a chiral center
   to form a third compound according to formula III

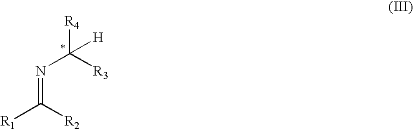

in which formula
R₁, R₂, R₃, R₄ and * are as defined above,
   whereby the compound according to formula (III) is subsequently reduced and thereby converted into a compound according to formula IV

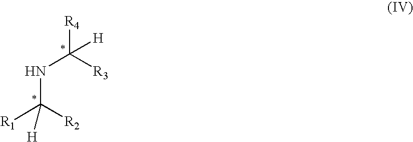

The process according to the invention is suitable for industrial production, i.e. production on large scale. An additional advantage is that this process does not require use of air and moisture sensitive reagents, or reagents that are expensive. Moreover the process according to the invention is less complex due to the lower number of process steps.

Compounds according to formula I are ketones wherein R₁ is a cycloalkyl group, and R₂ is a (cyclo)alkyl group, (cyclo)alkenyl group, aryl group, cyclic or acyclic heteroalkyl group or heteroaryl group. Optionally the R₂ group may contain one or more N, O, P or S atoms. If so desired, the R₂ group may be monosubstituted or polysubstituted with for example halogen, in particular chlorine or bromine, a hydroxy group, an alkyl or (hetero)aryl group with for example 1-10 carbon atoms and/or an alkoxy group or acyloxy group with for example 1-10 carbon atoms. Furthermore R₁ should not equal R₂ in order to obtain chiral products.

Preferably R₁ is a cycloalkyl group with 3 to 20 carbon atoms, more preferably a cycloalkyl group with 3 to 8 carbon atoms. Most preferably R₁ is a cycloalkyl group with 3 to 6 carbon atoms. In the process according to the invention this gives a high yield of the compound according to formula IV.

Preferably R₂ comprises 1 to 20 carbon atoms, more preferably 1 to 8 carbon atoms, and most preferably 1 to 3 carbon atoms. In the process according to the invention this gives a high yield of the compound according to formula IV.

Particular preferred compounds according to formula I are cyclohexyl methyl ketone, cyclopentyl methyl ketone and cyclopropyl methyl ketone. Compounds according to formula IV are very suitable as intermediates for the production of pharmaceutical or agrochemically active compounds.

Compounds according to formula II are chiral compounds wherein R₃ is an alkyl group, and R₄ is a substituted or unsubstituted phenyl- or naphthyl-group.

Preferably R₃ is an alkyl group with 1 to 6 carbon atoms, more preferably an alkyl group with 1 to 3 carbon atoms, most preferably R₃ is methyl. In the process according to the invention this gives a high yield of a compound according to formula IV.

If so desired, the phenyl- or naphthyl-group of R₄ may be monosubstituted or polysubstituted with for example halogen, in particular chlorine or bromine, a hydroxy group, an alkyl or (hetero)aryl group with for example 1-10 carbon atoms and/or an alkoxy group or acyloxy group with for example 1-10 carbon atoms.

A particular preferred compound according to formula II is a compound where R₃ is a methyl-group and R₄ is a phenyl-group, hereinafter referred to as phenyl ethyl amine (PEA). An advantage of PEA is that it gives a high diastereomeric excess of a compound according to formula IV in the process according to the invention. Furthermore PEA is a compound that is easily accessible.

Depending on the desired chirality of the compound according to formula IV, either an (R)- or (S)-configuration of the compound according to formula II may be chosen.

In the process according to the invention the compounds according to formula I and II are contacted, preferably in a solvent. In general solvents that form an azeotropic mixture with water are used. Suitable solvents include for example toluene and isopropylacetate.

Optionally a catalyst may be used upon contacting the compounds according to formula I and II. Preferred catalysts include acids, such as for example p-toluenesulphonic acid, or Lewis acids such as for example titaniumtetrachloride or titaniumtetraisopropoxide.

The temperature at which the compounds according to formula I and II are contacted preferably is between 0-140° C., more preferably between 20-120° C.

Upon contacting the compounds according to formula I and formula II in the process according to the invention a reaction mixture comprising compound III is formed. Said compound III is subsequently reduced into a compound IV. The reaction mixture comprising compound III may be purified before the subsequent reduction, however preferably compound III is directly converted into compound IV. Reduction of compound III can be effected for example with the aid of NaBH₄, LiAlH₄, or with hydrogenation catalysts, for example Pd, Pt or Raney-Ni, in combination with H₂. Especially reduction through NaBH₄ or Pd/H₂ was found to be very suitable, since this leads to high diastereoselectivities. Moreover NaBH₄ or Pd/H₂ was found to give a good yield even in the case when the compound according to formula III is substituted with low cycloalkyl groups, i.e. R₁ is cyclopropyl or cyclobutyl.

Reduction preferably is done at temperatures between 0 and 80° C. Advantage of this temperature range is that fast reduction is obtained. More preferably reduction is done at temperatures between 20 and 60° C. This leads to high diastereoselectivities.

From literature it is known that phenylethylamine derivatives are generally not crystalline but usually are oils and cannot easily be purified to diastereomerically pure compounds via, for example, recrystallization of salts thereof. Consequently such oil, whether or not derivatized, requires separation via for example. chromatography. Chromatography is not only an expensive technique but generally also leads to relatively low yields and consequently is less suitable for industrial production.

Surprisingly however it was found that salts of the compounds according to formula IV and for example an acid such as HCl, HBr, acetic acid and p-toluenesulphonic acid can be recrystallized in the case of incomplete diastereoselectivity, and that purification by means of a single crystallisation step often leads to at least 95% diastereomeric excess. Preferably the HCl salt of the compounds according to formula IV is recrystallized. This results in a very favourable diastereomeric excess upon a single recrystallization step.

Most particularly well prepared with the process according to the invention are diastereomeric compounds according to formula IV in which R₁ is cyclopropyl, cyclopentyl or cyclohexyl; R₂ comprises between 1 and 3 carbon atoms; R₃ is —CH₃ and R₄ is phenyl. These compounds can be obtained in high diastereomeric excess, as defined below, typically of at least 80 mol %. Moreover these compounds can be very well be recrystallized in one step, e.g. through stirring of a HCl salt of the compound according to formula IV in a solvent, for example in acetone or methyl-t-butylether, thereby reaching a diastereomeric excess of at least 98 mol %.

Diastereomeric excess (de) in this application is defined as the difference between the amounts of diastereomers divided by the sum of the amounts of the diastereomers, which quotient can be expressed as a percentage after multiplication by 100.

Furthermore enantiomeric excess (ee), as lateron used in this application, is defined as the difference between the amounts of enantiomers divided by the sum of the amounts of the enantiomers, which quotient can be expressed as a percentage after multiplication by 100.

The compounds according to formula IV, where R₁, R₂, R₃, R₄ and * are as previously defined, are novel compounds. The compounds preferably have a diastereomeric excess of at least 80%, in particular at least 90%, more particularly at least 98%. The compounds preferably have an enantiomeric excess of at least 80%, in particular at least 90%, more particularly at least 98%. The invention also relates to such compounds. With the process according to the invention compounds according to formula IV of (R,R), (R,S), (S,R), or (S,S) chirality can be obtained.

These compounds according to formula IV can be used as intermediates for pharmaceutical and agrochemically active compounds, for example cyclopropyl derivatives that may be used as antipsychotic agents and in agents for neuropsychiatric disorders.

The diastereomeric compounds according to formula IV may subsequently be converted into a corresponding chiral cycloalkyl amine by means of for example hydrogenolysis with H₂ using for example Pd as a catalyst. Through hydrogenolysis the chiral centre comprising R₃ and R₄ is split off from the compound according to formula IV, resulting in the corresponding chiral cycloalkyl amine according to formula V.

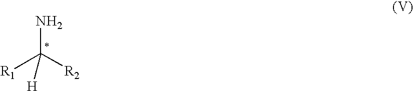

with R₁ and R₂ as previously defined.

Temperature during hydrogenolysis is chosen preferably between 0 and 40° C., more preferably between 20 and 30° C. This results in a high yield of chiral cycloalkyl amines.

A process for the preparation of chiral cyclopropylamines is known from Vogel, Roberts, J. Am. Chem. Soc 1966, 88, pages 2262-2271. The process as disclosed by Vogel yields racemic cycloalkylamines that are subsequently enantiomerically enriched by resolution processes. For example racemic cyclopropylethylamine is resolved through recrystallization as a salt of D-tartaric acid. Drawback however is that six recrystallizations of the salt of cyclopropylethylamine and D-tartaric acid were required to obtain enantiomerically pure cyclopropylethylamine. Another drawback is that the overall yield from the racemic amine to the enantiomerically enriched (R)-cyclopropylethylamine is only 15%.

The invention will now be further elucidated with the following examples, without being limited hereto.

### EXAMPLE Ia

### Synthesis of a Compound According to Formula III from (R)-Phenylethylamine and Cyclopropylmethylketone

To 250 ml toluene were successively added, 17.8 g (212 mmol) cyclopropylmethylketone, 27.7 g (237 mmol) (R)-phenylethylamine and 1 g (5.3 mmol) p-toluene sulphonic acid. The mixture was heated with stirring during 10 hours to reflux for azeotropic removal of water. Samples were taken and analyzed by GC.

Obtained was a solution of compound III (often also referred to as Schiff's base of (R)-phenylethylamine and cyclopropylmethylketone), in toluene. The molar ratio of the Schiff's base:(R)-phenylethylamine was 82:18.

The obtained solution could be used during the subsequent reduction step without need for isolation or purification.

### EXAMPLE Ib

### Reduction of the Schiff's Base of (R)-phenylethylamine and Cyclopropylmethylketone with NaBH₄ to Form a Compound According to Formula IV

To 250 ml of methanol was slowly added, while stirring, 3.0 g (79 mmol) NaBH₄. Then 50 ml of the solution as obtained in example Ia (containing about 29 mmol of compound III) was added in about 1 hour while keeping the temperature at ca 20-25° C. Then the so obtained mixture was stirred during 30 minutes. About 5 ml of H₂O was added slowly, followed by addition of 4N HCl until pH=1. Obtained was a system with a methanol/water phase and a toluene phase. The methanol in the methanol/water phase was removed under vacuum. The toluene phase was separated. The pH of the water phase was increased from 1 to about 11 with 10% NaOH/H₂O. The water phase was extracted 2 times with 50 ml diethylether. The two diethylether-extracts were combined and hereto was added 50 ml of a solution of HCl in methanol (prepared by adding 5 mL acetylchloride to 50 ml methanol). The methanol was evaporated and the residue was stirred in 50 ml acetone. The solid was filtered, washed with 2×5 ml acetone and dried until constant weight. Yield 3.3 g HCl salt of the compound according to formula IV. ¹H-NMR and GC revealed a ratio of 98.5:1.5 for the two diastereomers.

The free base of compound IV, in quantitative yield, was prepared by addition of 10% NaOH to the HCl salt of compound IV followed by extraction with EtOAc.

The overall yield is 41% for the two steps as described in examples Ia and Ib.

### EXAMPLE Ic

### Hydrogenolysis of Amine Obtained in Example Ib: Synthesis of Cyclopropylethylamine

An amount of 350 mg of the free base of compound IV as obtained from Example Ib was dissolved in 5 ml ethanol, whereto 100 mg 5% Pd/C (Engelhard ESCAT 142, 50% wet) was added. The mixture was hydrogenated at 3.5 bar H₂ for 30 h at 25° C. After filtration of the Pd/C with washing of the catalyst, a few drops of concentrated HCl were added to the filtrate. After evaporation of the ethanol 5 ml acetone was added which gave a white solid. After filtration and drying until constant weight 207 mg cyclopropylethylamine.HCl was obtained: yield 92%, ee>97%.

### EXAMPLE 2

### Preparation of Cyclopropylisobutylamine

Following similar procedures as described in Example 1, from cyclopropyl isopropyl ketone, cyclopropylisobutylamine can be obtained.

The required ketone can be obtained via methods described in the literature (see J. Am. Chem Soc, 1968, 90, 3766-3769.

### EXAMPLE 3

### Preparation of Cyclopropyl Heptyl Amine

Following similar procedures as described in Example 1, from cyclopropyl hexyl ketone, cyclopropyl heptyl amine can be obtained.

The required ketone can be obtained via methods described in the literature (see Tet Let, 2003, 44, 7175-7177

### EXAMPLE 4

### Preparation of 1-cyclopropyl 1-phenyl methylamine

Following similar procedures as described in Example 1, from the commercial available cyclopropyl phenyl ketone, the corresponding 1-cyclopropyl 1-phenyl methylamine can be obtained.

### EXAMPLE 5

### Preparation of 1-cyclopropyl 1-(4-fluorphenyl)methylamine

Following similar procedures as described in Example 1, from the commercial available cyclopropyl 4-fluorophenyl ketone, the corresponding 1-cyclopropyl 1-(4-fluorphenyl)methylamine can be obtained.

### EXAMPLE 6

### Preparation of 1-cyclopropyl 1-(thienyl)methylamine

Following similar procedures as described in Example 1, from the commercial available cyclopropyl thienyl ketone, the corresponding 1-cyclopropyl 1-(thienyl)methylamine can be obtained

1. Process for the preparation of a diastereomerically enriched compound, wherein a first compound according to formula (I) 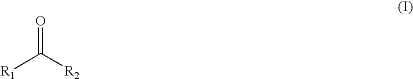
in which formula
R₁=a cycloalkyl group whereby R₁≠R₂
R2=a substituted or unsubstituted: (cyclo)alkyl group, (cyclo)alkenyl group, aryl group, cyclic or acyclic heteroalkyl group or heteroaryl group,
is contacted with a second compound according to formula (II) 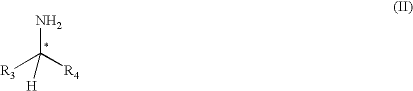
in which formula
R₃=an alkyl group
R₄=a substituted or unsubstituted phenyl- or naphthyl-group
*=a chiral center to form a compound according to formula (III) 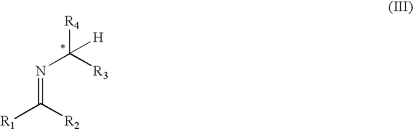
whereby the compound according to formula (III) is subsequently reduced and thereby converted into the diastereomerically enriched compound according to formula (IV)

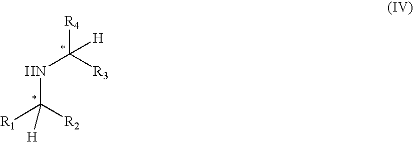

2. Process for the preparation of an enantiomerically enriched compound of formula (V) 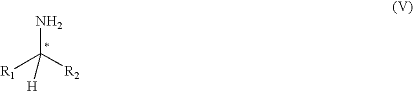
wherein
R₁=a cycloalkyl group whereby R₁≠R₂
R₂=a substituted or unsubstituted: (cyclo)alkyl group, (cyclo)alkenyl group, aryl group, cyclic or acyclic heteroalkyl group or heteroaryl group,
by contacting a first compound according to formula (I) 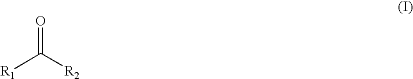
wherein R₁ and R₂ have the meaning given above, with a compound of formula (II) 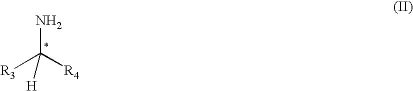
wherein
R₃=an alkyl group
R₄=a substituted or unsubstituted phenyl- or naphthyl-group
*=a chiral center to form a third compound according to formula (III) 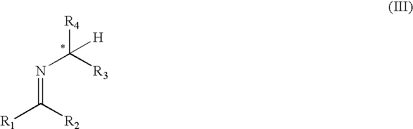
whereby the compound according to formula (III) is subsequently reduced and thereby converted into the diastereomerically enriched compound according to formula (IV) 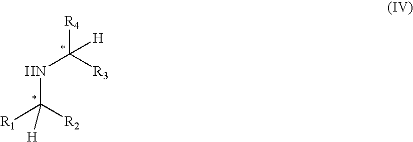
whereafter the compound according to formula (IV) is subsequently converted through hydrogenolysis into the enantiomerically enriched compound according to formula (V).

3. Process according to claim 1, wherein the compound according to formula (II) is (R)- or (S)-phenyl ethyl amine.

4. Process according to claim 1 wherein R₁ is cyclopropyl, R₂ is alkyl, R₃ is methyl and R₄ is phenyl.
